(19) United States
(12) Patent Application Publication
(10) Pub. No.: US 2024/0119314 A1
(43) Pub. Date: Apr. 11, 2024

(54) GENE CODING BREEDING PREDICTION METHOD AND DEVICE BASED ON GRAPH CLUSTERING

(71) Applicant: ZHEJIANG LAB, Hangzhou (CN)

(72) Inventors: Jingsong LV, Hangzhou (CN); Hongyang CHEN, Hangzhou (CN); Hao WANG, Hangzhou (CN); Xianzhong FENG, Hangzhou (CN)

(21) Appl. No.: 18/454,036

(22) Filed: Aug. 22, 2023

Related U.S. Application Data
(63) Continuation of application No. PCT/CN2022/121174, filed on Sep. 26, 2022.

(51) Int. Cl. G06N 5/022; G16B 20/00; G16B 40/20; G16B 45/00

(52) U.S. Cl. CPC G06N 5/022; G16B 20/00; G16B 40/20; G16B 45/00

(57) ABSTRACT

A method and a device for predicting gene coding breeding based on graph clustering. According to the present disclosure, a gene map is constructed based on inter-gene correlation strength; the gene map is subjected to clustering solution to obtain a number of co-regulated genomes and a genome cluster number information of each gene; gene allelic information and genome cluster number information are fused to obtain the gene cluster code of the sample; based on gene cluster code information and biological phenotype information to be predicted, a deep convolutional neural network is constructed to optimize the prediction performance of genetic breeding.

### CROSS-REFERENCE TO RELATED APPLICATIONS

The present application is a continuation of International Application No. PCT/CN2022/121174, filed on Sep. 26, 2022, the content of which is incorporated herein by reference in its entirety.

### TECHNICAL FIELD

The present disclosure mainly relates to the field of genetic breeding prediction of precise molecular breeding for crops, and mainly relates to a method and a device for gene coding breeding prediction based on graph clustering.

### BACKGROUND

With the development of gene sequencing technology, experimental technicians can obtain large-scale multi-sample gene data information with data mining application value based on sequencing, PCR (polymerase chain reaction), gene chip and optical atlas by wet experimental processes such as sample collection, library preparation and sequencing. After sequencing a whole genome, the accuracy of genome prediction model is very low. For example, soybean contains about 60 thousand genes, of which 40 thousand pairs of genes have 80 million mutations. However, the phenotype of genotype prediction can only be described qualitatively, but not analyzed quantitatively. This greatly limits the quantity, speed and quality of the crop breeding, especially the improvement of yield.

In order to improve the accuracy of molecular breeding, the current gene prediction methods for crop phenotype mainly include traditional statistical analysis methods such as Bayesian method, linear regression, and ridge regression. However, the deep learning methods that have achieved great success in the fields of voice, images and natural language cannot achieve greatly effects because of the shortage of samples in crop breeding. On the other hand, the dimension of gene data is very high, and it is difficult for traditional statistical analysis methods to quickly extract effective features from such high-dimensional gene feature data by using feature selection methods. It can be seen that the existing popular methods cannot solve the high-dimensional few sample problem in the crop molecular breeding.

In order to deal with the high-dimensional few sample problem in the crop molecular breeding, it is necessary to put forward an innovative genetic breeding prediction method to solve the problem of feature selection and extraction of high-dimensional features and the problem of insufficient coding of gene map features in complex model samples.

### SUMMARY

In view of the shortcomings of the prior art, the present disclosure provides a method and an device for gene coding breeding prediction based on graph clustering. By utilizing the interaction relationship network between genes contained in the gene map, the 15 cluster information of co-regulated genomes is extracted through graph clustering, and a new gene cluster coding mode of fusing gene allelic information and gene map cluster information is proposed, the features of regulatory genes used to control the output of biological phenotype are effectively extracted by using weight sharing by a deep convolution neural network to solve the problem that the classical model input coding layer has insufficient coding on the gene interaction relationship between the gene maps, and ensures the accuracy of genetic breeding prediction of the biological phenotype.

In order to achieve the above purpose, the present disclosure provides the following technical solution:

The present disclosure discloses a gene coding breeding prediction method based on graph clustering, including the following steps:

Acquiring genotype data and gene position information of an offspring to be predicted. Constructing an undirected graph as a gene map based on an inter-gene correlation strength in the genotype data.

Performing clustering solution on the gene map to obtain a number of co-regulated genomes and a genome cluster number of each gene.

Fusing allele information and genome cluster number information corresponding to each gene in the genotype data, and connecting in series to obtain a gene cluster code of a sample.

Inputting the gene cluster code and gene position information into a gene coding breeding prediction model to obtain the biological phenotype information of the offspring to be predicted; and screening a quality seed set based on the biological phenotype information of the predicted offspring.

The gene coding breeding prediction model is obtained by training based on a collected data set, and each sample data of the data set includes the gene cluster code, the gene position information and the biological phenotype information of the sample.

In an embodiment, the biological phenotype information includes measurable information such as quantity, quality, percentage and classification related to the target phenotype, and the allele information to be encoded includes SNP alleles, such as homozygous 0/0, 1/1 and heterozygous 0/1.

In an embodiment, the clustering solution is performed on the gene map to obtain the number of co-regulated genomes and the genome cluster number information of each gene, including:

Estimating the number of co-regulated genomes based on the spatial distribution features of the gene map to obtain a number of gene clustering clusters.

Calculating an intra-class distance and an inter-class distance for each gene according to the estimated number of the gene clustering clusters to determine a cluster to which the gene belongs.

Giving each gene clustering cluster unique cluster number information as the genome cluster number of each gene in a corresponding gene clustering cluster after clustering.

In an embodiment, the inter-gene correlation strength is obtained by calculating the similarity of multiple SNP loci strings of every two genes in the genotype data; the common method includes Pearson correlation coefficient, Spearman correlation coefficient, Euclidean distance, cosine similarity, Manhattan distance, Hamming distance, editing distance and the like; and the adjacent edge weight is generally expressed by the correlation between genes or normalized values thereof.

In an embodiment, a method for gene clustering includes spatial clustering (Kmeans, etc.), density clustering (DBSCAN, etc.), hierarchical clustering (bottom-up method and top-down method) or spectral clustering, etc.

In an embodiment a method for estimating the number of co-regulated genomes comprises a statistical method, a random method, an exhaustive method, an iterative method and the like; and the iterative method mainly refers to a clustering number method determined by bottom-up or top-down iterative clustering in hierarchical clustering.

In an embodiment, among them, the spectral clustering method mainly uses the connected components of Laplacian matrix and other computational graphs for clustering; the calculation methods of intra-class distance and inter-class distance include the preferred gene similarity calculation method as mentioned above, and the intra-class and inter-class distances defined by graph connectivity and neighborhood features.

In an embodiment, the gene cluster number information is given by a clustering method, or in a random way or a sequential way.

In an embodiment, the fusion mode of allele information and genome cluster number information is string concatenation.

In an embodiment, the structure of the gene coding breeding prediction model includes modules such as an input layer, an embedding layer, a convolution layer, a pooling layer, a fully-connected layer and an output layer of gene cluster code, and strategies to improve the generalization ability of a neural network, including L1/L2 regularization, Dropout, etc., and optimization learning algorithms include Adam, etc.

In an embodiment, the input layer includes the gene cluster code information obtained in step 4, or the gene cluster code information is added with the gene position information, and the output layer includes the classification layer or regression layer related to the target task, or serves as a pre-trained multi-task classification and regression layer.

In an embodiment, the gene coding breeding prediction model is obtained by two-phase learning and training; in the first phase of learning, as a pre-trained twin network, it receives coding inputs from two gene strings, and simultaneously learns difference tasks and addition tasks at the output layer; in the second phase of learning, as the pre-fixed weight network layer for subsequent training, it participates in the fine-tuning learning of the target task.

In an embodiment, the method of screening quality seed sets is obtaining the optimal seed sets and the corresponding parent combinations thereof by setting and optimizing reasonable thresholds.

Compared with the prior art, the gene coding breeding prediction method based on graph clustering has the following beneficial effects: firstly, the information of biological phenotype to be predicted and allele information to be coded required by accurate molecular breeding are collected; then, the gene map and the adjacent edge weight are determined based on the inter-gene correlation strength; then, the gene map is subjected to clustering solution to obtain the number of co-regulated genomes and the genome cluster number information of each gene; then, the gene allele information and genome cluster number information are fused to obtain the gene cluster code of the sample; finally, based on gene cluster code information, or additional gene position information and biological phenotype information to be predicted, a deep convolutional neural network is constructed to optimize the prediction performance of genetic breeding; this method makes full use of the inter-gene interaction relationship network contained in gene map, extracts the cluster information of co-regulated genomes through graph clustering, and proposes a new gene cluster code mode combining gene allelic information and gene map clustering information, as well as additional gene position information; in addition, by using the weight sharing of the deep convolutional neural network, the method can effectively extract the regulatory gene features used to control the output of biological phenotypes, solve the problem that the classical model input coding layer has insufficient coding on the gene interaction relationship between gene maps, and ensure the accuracy of genetic breeding prediction of biological phenotypes.

### BRIEF DESCRIPTION OF DRAWINGS

FIG. 1 is a flow chart of a gene coding breeding prediction method based on graph clustering provided by an embodiment of the present disclosure;

FIG. 2 is a schematic diagram of cloud-side collaborative deployment of a gene coding breeding prediction device based on graph clustering provided by an embodiment of the present disclosure;

FIG. 3 is a flowchart for collecting gene information provided by an embodiment of the present disclosure;

FIG. 4 is an architecture diagram of a single-phase deep convolution neural network model provided by an embodiment of the present disclosure;

FIG. 5 is an architecture diagram of a two-phase deep convolution neural network model provided by an embodiment of the present disclosure; and

FIG. 6 is a structural block diagram of a gene coding breeding prediction device based on graph clustering provided by an embodiment of the present disclosure.

Reference signs: information collection module 11; gene clustering module 12; coding pre-training module 13; subsequent training module 14; breeding prediction and screening module 15.

### DESCRIPTION OF EMBODIMENTS

In order to make the purpose, technical solution and advantages of the present disclosure more clear, the present disclosure will be further explained in detail through the attached drawings and examples. However, it should be understood that the specific embodiments described herein are only for explaining the present disclosure, and are not used to limit the scope of the present disclosure. In addition, in the following description, descriptions of well-known structures and technologies are omitted to avoid unnecessarily confusing the concepts of the present disclosure.

In order to facilitate the understanding of this embodiment, firstly, a gene coding breeding prediction method based on graph clustering disclosed in this embodiment of the present disclosure is introduced in detail.

### EXAMPLE 1:

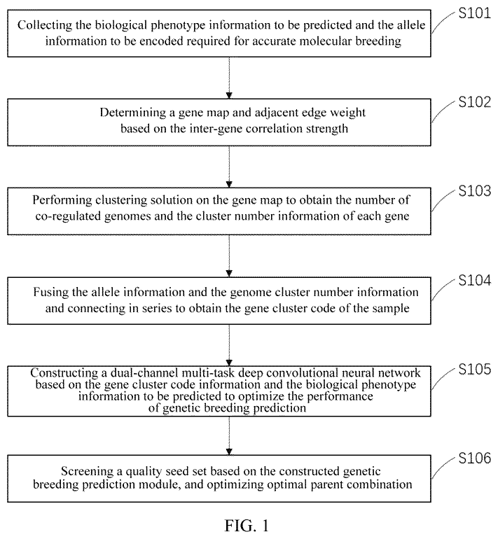

The present disclosure relates to a gene coding breeding prediction method based on graph clustering, which includes coding genes by graph clustering, and then predicting through a gene coding breeding prediction model to obtain the biological phenotype information of the offspring to be predicted; screening quality seed sets based on the biological phenotype information of the predicted offspring. The gene coding breeding prediction model is obtained by training based on the collected data set, and the training method, referring to FIG. 1, specifically includes the following steps.

S101, the biological phenotype information, genotype data and gene position information of each sample required for accurate molecular breeding are collected to construct a data set.

In the embodiment of the present disclosure, the executive body of the method is a computational breeding center. In an embodiment, if the computational breeding center is set on the computational cloud, then the computational cloud is the executive body of the method; if the computational breeding center is set on the computational node, then the computational node is the executive body of the method.

In the embodiment of the present disclosure, the computational breeding center predicts the biological phenotype based on the high-dimensional genotype data, aiming at screening the male parent and the female parent based on the predicted value, so as to generate excellent offspring. However, ordinary gene coding, mainly based on numerical value, numerical mapping or One-hot Encoding, cannot effectively extract the multi-neighborhood structural features in gene network maps. Gene coding based on graph clustering can effectively extract this structural information, thus improving the ability of breeding prediction.

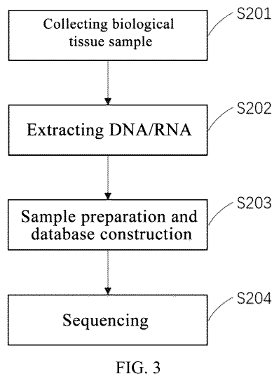

In an alternative embodiment, referring to FIG. 3, the collection of phenotypic and genotypic information required for breeding prediction includes: collecting biological tissue samples, extracting DNA/RNA, preparing a sample database and sequencing.

S102, an undirected graph is constructed as a gene map for the genotype data of each sample based on the strength of inter-gene correlation; the strength of inter-gene correlation can be obtained by calculating the similarity of multi-SNP locus strings of every two genes in the genotype data, and the methods include Pearson correlation coefficient, Jaccard correlation coefficient, Spearman correlation coefficient, Euclidean distance, cosine similarity of included angles, Manhattan distance, Hamming distance, editing distance, Chebyshev distance, Minkowski distance, information entropy and the like; the calculated 5 similarity is the adjacent edge weight of the undirected graph. As an embodiment, the inter-gene correlation strength is determined by the Pearson correlation coefficient, and the calculation formula is as follows:

$$S i m ( X , K ) = \frac{{\sum }_{i = 1}^{N} ( {X}_{i} - \overline{X} ) ( {K}_{i} - \overline{K} )}{( \sqrt{{\sum }_{i = 1}^{N} {( {X}_{i} - \overline{X} )}^{2}} ) ( \sqrt{{\sum }_{i = 1}^{N} {( {K}_{i} - \overline{K} )}^{2}} )}$$

where X and K are vector representations of two genes, X₁ and K₁ are the i^{th} components of the vector representations of genes X and K, respectively, and X̅ and K̅ are the mean values of corresponding genes. N is the dimension of a gene vector, in which the adjacent edge weight is the part from 0 to 1 in the inter-gene correlation strength, and the value that is not in the interval is 0, which means that there is no connected edge.

In an alternative embodiment, based on the gene string information and the gene correlation calculation formula of the development centralized sample, the correlation between gene loci is calculated, and the correlation heat map between gene loci, that is, the adjacency edge weight and adjacency matrix of gene loci, is obtained.

S103, the gene map constructed in step S102 is subjected to clustering solution to obtain the number of co-regulated genomes and the cluster number information of each gene.

After constructing a gene map, unsupervised clustering is performed on the map. The gene clustering method includes spatial clustering, density clustering, hierarchical clustering or spectral clustering, etc. In an embodiment y, this step includes the following sub-steps:

The number of co-regulated genomes, that is, a number of gene clusters, is estimated based on the spatial distribution features of the gene map; the method for estimating the number of co-regulated genomes is a statistical method, a random method, an exhaustive method or an iterative method, and the iterative method mainly refers to a cluster number method determined by bottom-up or top-down iterative clustering in hierarchical clustering.

An intra-class distance and an inter-class distance are calculated for each gene according to the estimated number of gene clusters to determine a cluster to which the gene belongs;

After clustering, each gene cluster is given unique cluster number information as the genome cluster number of each gene in a corresponding gene cluster; and the gene cluster number information is given by the clustering method itself, or in a random way or a sequential way.

In an alternative embodiment, based on the gene map corresponding to the adjacency matrix of gene loci constructed above, the connected subgraph algorithm is applied to obtain the clustering result of gene loci, and the isolated gene points are connected together and numbered in sequence to obtain the number of co-regulated genomes and the genome cluster number information of each gene.

S104, the allele information corresponding to each gene in the genotype data and the genome cluster number information are fused in series to obtain the gene cluster code of the sample.

After obtaining the gene cluster number information, the fused gene cluster code of the sample can be obtained by concatenating elements one by one. The coding mode of gene clustering is as follows:

Encode(Sᵢⱼ)=SNP(Sᵢⱼ) ⊕ Group(Sᵢⱼ)

where Sᵢⱼis the gene of the j^{th} component of a sample i, SNP(Sᵢⱼ) is the corresponding allele feature, that is, the code of the original gene in the genotype data, Group(Sᵢⱼ) is the corresponding cluster number, ⊕ is a fusion operator and represents the symbol concatenation operation.

S105, based on gene cluster code information, gene position information and biological phenotype information, a gene coding breeding prediction model is constructed and trained to optimize the performance of genetic breeding prediction.

After the gene cluster code of the sample is completed, the gene cluster code features of the sample can be obtained. Based on the features, the gene position information and the biological phenotype information to be predicted can be added to construct the gene coding breeding prediction model based on the deep convolutional neural network, and the Dropout and L1/L2 regularization strategy optimization can be added to optimize the genetic breeding prediction performance.

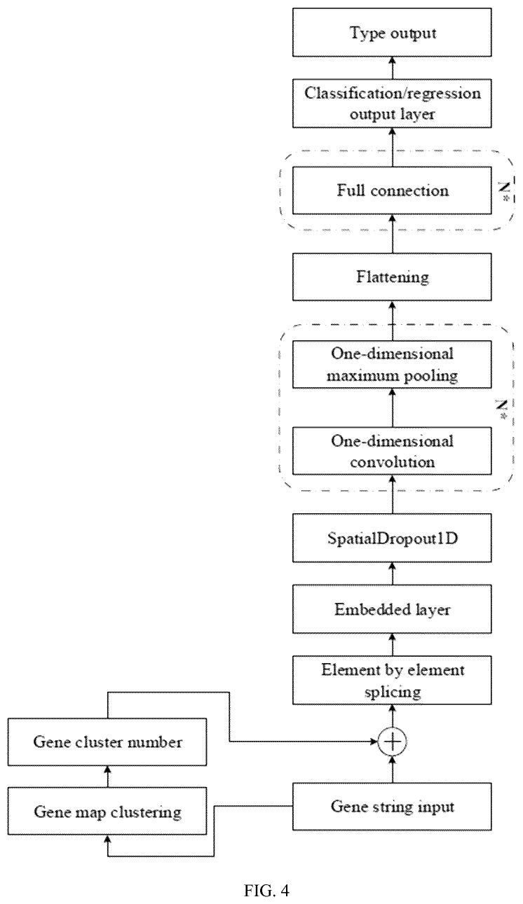

In an alternative embodiment, referring to FIG. 4, the input and coding layer (gene cluster code input layer) of a deep convolutional neural network is constructed by fusing allele information and gene cluster number information by splicing elements by elements, and then the embedded layer, SpatialDropoutlD, one-dimensional convolution, one-dimensional maximum pooling, flattening, full connection, and finally the classification/regression output layer and phenotypic output related to the target task are connected in sequence.

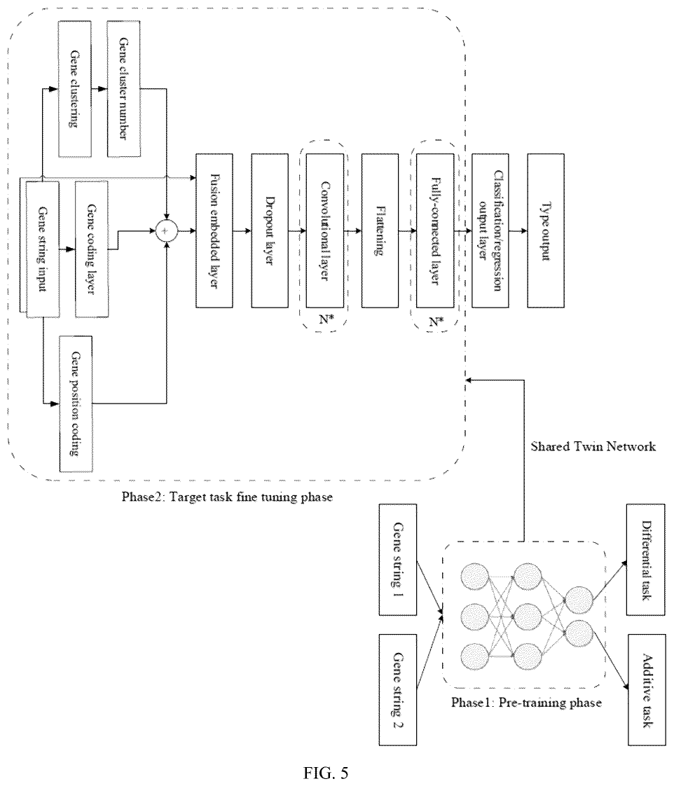

In an alternative embodiment, referring to FIG. 5, the model construction and learning phases are divided into two phases: the first phase is the pre-training phase, in which the input and coding layer of a dual-channel multi-task deep convolutional neural network is formed by fusing gene allele information, gene cluster number information and gene position 15 information, and then the deep convolutional neural network as a shared twin network is connected, and the output layer is connected with the difference task and the addition task. The input of the whole network is left and right channels, that is, the input of gene strings with two different samples, and the output is multi-task output. The difference task is to determine the positive and negative polarity of the difference between the phenotypic values 20 corresponding to the left and right channel gene strings, and the addition task is a regression task of the sum of the phenotypic values corresponding to the left and right channel gene strings. The target loss functions of the difference task and the addition task are L⁻ and L⁺ respectively. Generally speaking, L⁺ may be the mean square error MSE and L⁻ may be the cross entropy, as follows:

$${L}^{-} = \frac{1}{M} {\sum }_{l = 1}^{M} - [ I ( {Y}_{i 1} \geq {Y}_{i 2} ) log ( σ ( {i 1}_{} - {i 2}_{} ) ) + ( 1 - I ( {Y}_{i 1} \geq {Y}_{i 2} ) ) log ( 1 - σ ( {i 1}_{} - {i 2}_{} ) ) ]$$

$${L}^{+} = \frac{1}{M} {\sum }_{i = 1}^{M} {( {Y}_{i 1} + {Y}_{i 2} - - )}^{2}$$

where Yᵢ₁ and Yᵢ₂ are the actual sample label values, and [symbol] and [symbol] are the sample predicted values, and I(X) is the indicative function; the function value is 1 when X is true and 0 when X is false; A4 - Indicates the number of samples; σ is a Sigmoid function; the calculation formula is as follows:

$$σ ( x ) = \frac{1}{1 + {e}^{- x}}$$

where e is a natural constant, and its value is approximately equal to 2.71828.

The multi-task target loss function of the first phase is as follows:

L₁=αL⁺+βL⁻

where, L⁺, L⁻ and I are the loss functions of the addition task, the difference task and the total task respectively, α and β are the weight hyperparameters of the loss functions of the addition task and the difference task respectively, and the parameter values can be determined by the grid search method.

The second phase is the subsequent training phase. By loading the pre-trained shared twin network in the first phase and fixing the network weight so that it will not participate in the network weight optimization in the subsequent training phase, and then connecting the classification/regression output layer and phenotypic output related to the target task, the deep convolutional neural network in the subsequent training phase is constructed. Based on the gene string sample data and the corresponding phenotypic data in the development set, the model of the subsequent training phase of the target is learned and optimized, so as to obtain the target prediction model which is finally used in the breeding task. For the phenotypic regression prediction task, the target loss function in the second phase takes the mean square error MSE; for the phenotypic classification and prediction task, the target loss function of the second phase takes cross entropy. Taking the regression forecasting task as an example, the target loss function of the second phase is as follows:

$${L}_{2} = \frac{1}{M} {\sum }_{i = 1}^{M} {( {Y}_{i} - )}^{2}$$

S106, quality seed sets are screened, that is, the optimal parent combination is optimized based on the constructed genetic breeding prediction model.

For a trait classification task, a seed pool predicted as the specified trait classification, i.e., the optimal parent combination, is directly screened based on the constructed genetic breeding prediction model; for the trait regression, that is, numerical prediction task, the optimal parent combination is the seed pool whose prediction value reaches or exceeds the specified threshold based on the constructed genetic breeding prediction model. For the trait classification task, the designated screening threshold can be obtained by comprehensive optimization of the screening ratio and experimental field size.

The existing coding methods of gene input features usually consider numerical format or One-hot format coding. For example, in a numerical format, 0/0, 0/1 and 1/1 are usually mapped to −1, 0 and 1, or to 0, 1 and 2, which cannot express complex gene network features. However, the One-hot coding only discretizes the original features and does not increase the information content of the features, while the gene coding based on graph clustering combines the original allele features and the co-regulated genome features, thus capturing the upper-level graph neighborhood structure features, which greatly improves the prediction ability of the model based on the deep neural network. This method makes full use of the inter-gene interaction relationship network contained in gene maps, extracts the cluster information of co-regulated genomes through graph clustering, and proposes a new gene cluster code mode combining allele information and gene map clustering information, and uses the weight sharing of a dual-channel multi-task deep convolution neural network to effectively extract the regulatory gene features used to control the output of biological phenotypes, thus solving the problem that the classical model input coding layer has insufficient coding on the gene interaction relationship between the gene maps, ensures the accuracy of genetic breeding prediction of the biological phenotype.

### EXAMPLE 2:

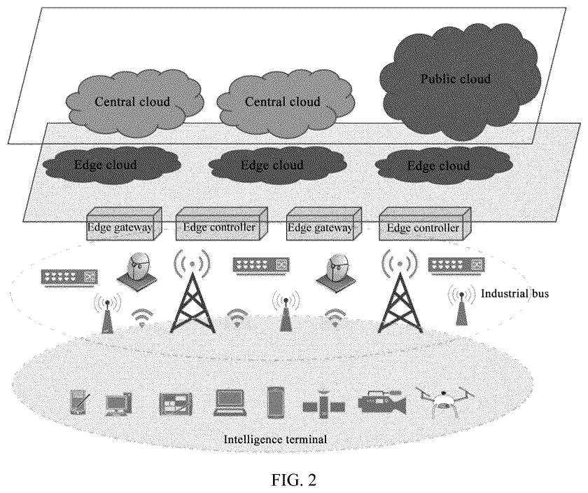

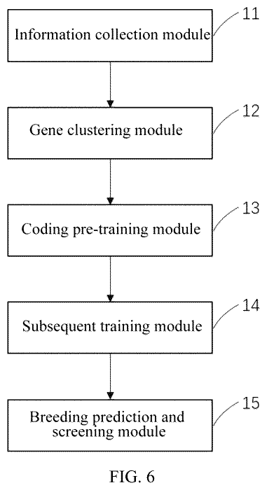

A gene coding breeding prediction device based on graph clustering, referring to FIGS. 2 and 6, includes an information collection module 11, a e gene clustering module 12, a coding pre-training module 13, a subsequent training module 14 and a breeding prediction and screening module 15.

The information collection module 11 is used for collecting the biological phenotype information and genotype data to be predicted required by accurate molecular breeding. This module is mainly completed by intelligent terminals.

The gene clustering module 12 is used to determine a gene map and the adjacent edge weight based on the inter-gene correlation strength, and perform clustering solution on the gene map to obtain the number of co-regulated genomes and the genome cluster number information of each gene. This module is completed by cloud edge collaborative computing.

The coding pre-training module 13 is used to fuse gene allelic information, genome cluster number information and gene position information, and construct a dual-channel multi-task deep convolution neural network based on the fused coding information, and simultaneously carry out learning training and network weight optimization on the differential task and the addition task for the dual gene string enhancement data, so as to obtain a shared twin network and its weight for subsequent training. This module is completed by cloud edge collaborative computing.

The subsequent training module 14 is used to load the shared twin network and its weights obtained by the coding pre-training module and solidify the weights, and perform subsequent training and optimization for the classification/regression task related to the breeding target traits to obtain a prediction model related to the breeding target traits. This module is completed by cloud edge collaborative computing.

The breeding prediction and screening module 15 is used to screen quality seed sets based on the genetic breeding prediction model constructed by the subsequent training module, that is, optimizing the optimal parent combination. This module is completed by cloud edge collaborative computing.

In the gene coding breeding prediction device based on graph clustering in the embodiment of the present disclosure, firstly, training set and prediction set data required for genetic breeding prediction are collected; the training set data includes biological phenotype information to be predicted and allele information to be encoded required for accurate molecular breeding, and the prediction set data includes allele information of seeds to be predicted; then, the correlation between different gene loci is calculated, a gene adjacency matrix and a map are constructed based on the strength of the correlation, and clustering solution is performed on the gene map to obtain the number of co-regulated genomes and the cluster number information of genomes of each gene; then, the gene allele information, genome cluster number information and gene position information are fused to construct a dual-channel multi-task deep convolutional neural network for pre-training, and the shared twin network and its weight are obtained; then, the shared twin network and its weights are loaded, and the weights are solidified, and the classification/regression tasks related to breeding target traits are continuously trained and optimized to obtain the prediction model related to breeding target traits; finally, based on the genetic breeding prediction model, quality seed sets are screened, that is, the optimal parent combination is optimized. The device can make full use of the inter-gene interaction relationship network contained in the gene map, extract the cluster information of co-regulated genomes through graph clustering, and propose a new gene cluster code mode combining the allele information of genes and the cluster information of the gene map. By using the weight sharing of the dual-channel multi-task deep convolution neural network, it can effectively extract the features of regulatory genes used to control the output of biological phenotypes, solve the problem that the classical model input coding layer has insufficient coding of the gene interaction relationship between gene maps, and ensure the accuracy of genetic breeding prediction of biological phenotypes.

The computer program product based on the gene coding breeding prediction method and device based on graph clustering provided by the embodiment of the present disclosure includes a computer-readable storage medium storing program codes, and the instructions included in the program codes can be used for executing the methods described in the previous method embodiments. The specific implementation can be seen in the method embodiments, so the details are not repeated here.

It can be clearly understood by those skilled in the art that for the convenience and conciseness of description, the specific working process of the system and device described above may refer to the corresponding process in the aforementioned method embodiment, and will not be repeated here.

The functions can be stored in a computer-readable storage medium if they are implemented in the form of software functional units and sold or used as independent products. Based on this understanding, the technical solution of the present disclosure can be embodied in the form of a software product, which is stored in a storage medium and includes several instructions to make a computer device (which can be a personal computer, a server, a network device, etc.) execute all or part of the steps of the method described in various embodiments of the present disclosure. The aforementioned storage media include: U disk, mobile hard disk, Read-Only Memory (ROM), Random Access Memory (RAM), magnetic disk or optical disk and other media that can store program codes.

The above is only the preferred embodiment of the present disclosure, and it is not used to limit the present disclosure. Any modification, equivalent substitution or improvement made within the spirit and principle of the present disclosure should be included in the protection scope of the present disclosure.

What is claimed is:

1. A gene coding breeding prediction method based on graph clustering, comprising:
acquiring genotype data and gene position information of an offspring to be predicted;
constructing an undirected graph as a gene map based on an inter-gene correlation strength in the genotype data;
performing clustering solution on the gene map to obtain a number of co-regulated genomes and a genome cluster number of each gene;
fusing allele information and genome cluster number information corresponding to each gene in the genotype data , and connecting the fused information in series to obtain a gene cluster code of a sample;
inputting the gene cluster code and gene position information into a gene coding breeding prediction model to obtain biological phenotype information of the offspring to be predicted; and
screening a quality seed set based on the biological phenotype information of the predicted offspring;
wherein the gene coding breeding prediction model is obtained by training based on a collected data set, and each sample data of the data set comprises the gene cluster code, the gene position information and the biological phenotype information of the sample.

2. The method according to claim 1, wherein the inter-gene correlation strength is obtained by calculating a similarity of multiple SNP loci strings of every two genes in the genotype data in a method comprising Pearson correlation coefficient, Jaccard correlation coefficient, Spearman correlation coefficient, Euclidean distance, cosine similarity of included angles, Manhattan distance, Hamming distance, editing distance, Chebyshev distance, Minkowski distance and information entropy; and the calculated similarity is used as an adjacent edge weight to construct the undirected graph.

3. The method according to claim 1, wherein said performing clustering solution on the gene map to obtain the number of co-regulated genomes and the genome cluster number information of each gene comprises:
estimating the number of co-regulated genomes based on a spatial distribution feature of the gene map to obtain a number of gene clustering clusters;
calculating an intra-class distance and an inter-class distance for each gene according to the estimated number of the gene clustering clusters to determine a cluster to which the gene belongs; and
giving the each gene clustering cluster unique cluster number information as the genome cluster number of each gene in a corresponding gene clustering cluster after clustering.

4. The method according to claim 3, wherein a method for gene clustering comprises spatial clustering, density clustering, hierarchical clustering or spectral clustering.

5. The method according to claim 3, wherein a method for estimating the number of co-regulated genomes comprises a statistical method, a random method, an exhaustive method or an iterative method, and wherein the iterative method comprises determining a clustering number method by bottom-up or top-down iterative clustering in hierarchical clustering.

6. The method according to claim 1, wherein the gene cluster number information is given by a clustering method itself, in a random way, or in a sequential way.

7. The method according to claim 1, wherein the biological phenotype information comprises quantity, quality, percentage or classification related to a target phenotype.

8. The method according to claim 1, wherein the gene coding breeding prediction model comprises an input layer, an embedding layer, a convolution layer, a pooling layer, a fully-connected layer and an output layer of the gene cluster code.

9. The method according to claim 1, wherein the gene coding breeding prediction model is obtained by two-phase training, and wherein a first phase based on a shared bridge network comprises a dual-channel gene cluster code input layer receiving gene cluster code inputs from two samples, respectively, and simultaneously learns difference tasks and addition tasks at an output layer; and a second phase based on fixed network parameters trained on the first phase only comprises a gene cluster code input layer accepting an input of the gene cluster code and the gene position information from one sample to participate in fine-tuning learning of a target task until the training is completed.

10. A gene coding breeding prediction device based on graph clustering, comprising a memory, a processor and a computer program, wherein the computer program is stored in the memory and runs on the processor, wherein the processor, when executing the computer program, implements the gene coding breeding prediction method based on graph clustering according to claim 1.
